(19) United States
(12) Patent Application Publication
(10) Pub. No.: US 2015/0133816 A1
(43) Pub. Date: May 14, 2015

(54) COMPLETE UPPER RESPIRATORY TRACT CLEANSING SYSTEM AND ITS USE

(71) Applicant: James Zhou Liu, Downingtown, PA (US)

(72) Inventor: James Zhou Liu, Downingtown, PA (US)

(21) Appl. No.: 14/076,313

(22) Filed: Nov. 11, 2013

(51) Int. Cl. A61M 1/00; A61B 10/00

(52) U.S. Cl. CPC A61M 1/0003; A61B 10/0045; A61B 10/0051; A61B 2010/0054; A61M 2202/0401; A61M 2202/0466; A61M 2202/0492; A61M 2210/0618; A61M 2210/0662; A61M 2210/0625
USPC 600/578; 604/540

(57) ABSTRACT

An extracting device for diagnosis and treatment of diseases includes a tip equipped with a one-way liquid valve; a chamber equipped with a one-way air valve; and an elastic bulb for generating suctioning-pushing pressure when it is squeezed and released. When the user squeezes and releases the elastic bulb, the liquid will be drawn into the chamber through the tip. Additional fluidal material can enter into the bulb. After the liquid or mucus is drawn out, the body function such as nasal breath can be resumed. A specimen can be taken into the chamber through the tip and then transferred to a test tube for laboratory diagnosis. A new method of obtaining and removing body secretion is provided. No any form of electricity is needed. The parts can be easily disassembled for clean for repeated uses as a mucus extractor.

### BACKGROUND OF THE INVENTION

1. Field of the Invention

The present invention relates to an extracting device, a process and a method for treatment and diagnosis of a disease, and more particularly as a liquid extracting device for easily drawing or removal of the liquid from a user or a patient. The device can be used to safely and easily draw liquid as a specimen for diagnose of a disease and also for removal of undesired liquid from the body as a mean to treat a disease.

2. Description of the Prior Art

The quantity and quality of the air a human takes into the lungs is affected to some degree by the amount of mucus, which obstructs the passages of the respiratory system. Also, when there is excess mucus congesting these passageways, there is also a greater possibility for the growth of microbes. Normally, a certain amount of mucus lining is necessary for the protection of the membranes of the respiratory and digestive systems. It serves as a fluid transportation system, which also carries microbes and foreign particles out of the body. Many times, however, people suffer from excessive build-up of mucus in their system. This leads to frequent colds, sinusitis and, in general, a phlegmatic condition with less energy and vitality.

Excessive mucus in the nose of an adult or an infant is typically caused by viral or bacterial infection, but allergies, medications and pregnancy are other possible triggers, according to the National Institutes of Health. While generally nothing more than a nuisance, chronic nasal congestion can interfere with sleeping and eating or lead to problems with speech and hearing in children. Adults and older children can remove mucus from the nose by performing nose irrigation with a saline solution and blowing. However younger children and babies are unable to blow their own noses, therefore leaving suction as the only option parents or caretaker can perform in order for baby mucus removal.

One currently available device includes Babbitt et al.'s invented portable device used to aspirate and remove fluids from nasal and sinus cavities (U.S. Pat. No 4,403,611 issued Sep. 13, 1983). However since this device relies on the propeller means rotatably mounted to a drive motor to generate the vacuum power, this device is neither light in weight nor portable for convenience. More importantly the fact that it lacks a pair of liquid and air valves to allow only one way flow of the liquid, can easily cause serious re-infect issue with back-flow possibility. In addition, its heavy weight and complicated manufacturing process in comparison would mean inconvenience and unaffordability to many consumers.

Another available method of administrating a pharmacological solution into the nasal cavity of a patient was described by Lofstedt (U.S. Pat. No. 5,116,311, issued May 26, 1992). Their device has a compressible container, which not only applies a negative pressure to aspirate and forces the solution flow into the nasal cavity, but also aspirates or re-infect user with the contaminated solution containing nasal secretions and other contaminants. It is therefore, not an ideal device for nasal mucus removal.

The other current devices for removing or drawing mucus from the nasal cavity are rubber bulb, syringe, or devices comprise a chamber having a nozzle formed or provided on one end thereof, for engaging into an inner space of the body, and for vacuuming and cleaning or removing the liquid from a user or a patient. The invented device is simple to manufacture and easy to use, with no requirements for batteries or electricity while some of the previously discussed devices generate vacuuming pressure with electricity or batteries. In general, these electronic devices are not easy to clean after each use and cost more for consumers to purchase.

Generally, using a rubber bulb to remove mucus in babies needs to perform a procedure with a number of steps, such as (1) lay the baby in the lap, with baby's head near the mother's knees and baby's feet against the mother's stomach. Angle baby's head back slightly so that the mother can easily see the baby's nostrils; (2) administer two to three drops of saline into each nostril using an eyedropper. Let the saline remain inside the nostrils for 5 to 10 seconds. This will loosen any dried mucus and make removal easier; (3) squeeze the bulb end of the bulb syringe to expel any air and create a vacuum, and then insert the tip of the bulb syringe into one nostril. Place the tip just inside the nostril; (4) release pressure on the bulb slowly to suction the mucus out of the baby's nose. Remove the bulb syringe and then squeeze it forcefully over a tissue to expel the mucus collected from the baby's nose; (5) repeat the process for the other nostril; (6) clean the bulb syringe between uses by filling it with warm soapy water, shaking it, and then rinsing it with clean water. Store it with the tip pointed downward to dry.

Therapeutic paracentesis is currently performed when the body fluid, such as ascites, has accumulated enough to cause respiratory compromise, abdominal pain, or worsening of existing inguinal or umbilical hernias. Most common causes of ascites are cirrhosis of the liver, cancer, and congestive heart failure. Paracentesis may be done to determine the cause of fluid buildup in the abdominal cavity, to diagnose infection in the peritoneal fluid, to detect certain types of cancer, such as liver cancer, to evaluate abdominal injury. Because the device for being used to perform paracentesis is either a catheter or a syringe, both are lack of anti-backflow mechanism, and the holding volume of a syringe is limited. There is a risk of infection, excessive bleeding or perforating a loop of bowel when a large needle/syringe is used. These risks can be minimized greatly with the use of ultrasound guidance. In a non-clinic or non-hospital environment, such as a battle field, there would be hard to have all the needed equipments for reducing the risk. There is a need to have a new device which can minimize the side effect of paracentesis, and the device should also have a large volume and be equipped with an anti-backflow system.

The present invention is to eliminate the afore-described disadvantages of these prior devices. It is simple but with important two-valve anti-backflow function and the sucking pressure that is well controllable by the user's hand.

### SUMMARY OF THE INVENTION

The objective of the present invention is to provide a device to safely and easily draw or remove the liquid from the body of a patient to be used to treat or diagnose a disease.

In accordance with one aspect of the invention, the extracting device comprising a unique cone-shaped tip to be easily inserted into a space to hold the liquid, like the mucus in the nose; a one-way liquid valve which is fixed in the tip to allow the liquid only flow in one direction, not back flow; a chamber which is connected to the cone-shaped tip serving as a container for the liquid; an air valve which is fixed in the chamber to release pressure when the liquid is entered into the chamber; an elastic bulb which is connected to the other end of the chamber. When squeezed or released, the elastic bulb generates suctioning or pushing air pressure to draw the liquid out of the body. The small amount of the withdrawn liquid can be used as specimen for a disease diagnosis in the clinical laboratory. The large amount of the withdrawn liquid can be used as a way to reduce the internal pressure to treat a disease.

### BRIEF DESCRIPTION OF THE DRAWINGS

FIG. 1 is an overall view of an assembled extracting device for diagnosis and treatment of diseases in accordance with the present invention.

FIG. 2 is an overall view of the elastic bulb of the extracting device for diagnosis and treatment of diseases in accordance with the present invention.

FIG. 3 is an overall view of the chamber of the extracting device for diagnosis and treatment of diseases in accordance with the present invention.

FIG. 4 is an overall view of the cone-shaped tip of the extracting device for diagnosis and treatment of diseases in accordance with the present invention.

FIG. 5 is an illustration when the user is using the extracting device to draw the liquid.

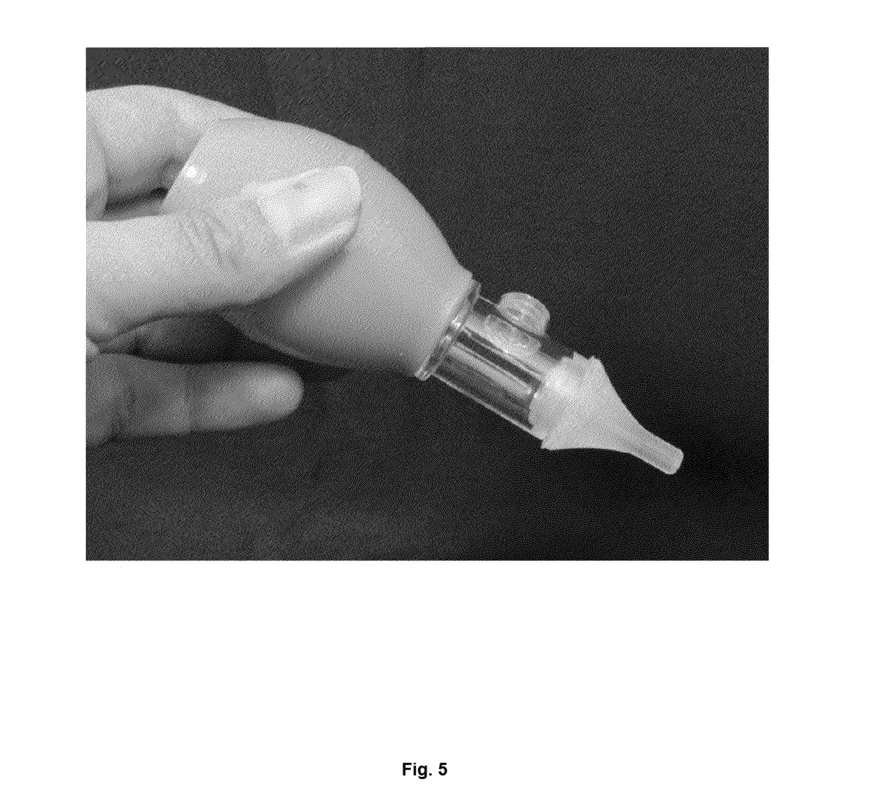

### DETAILED DESCRIPTION OF THE PREFERRED EMBODIMENT

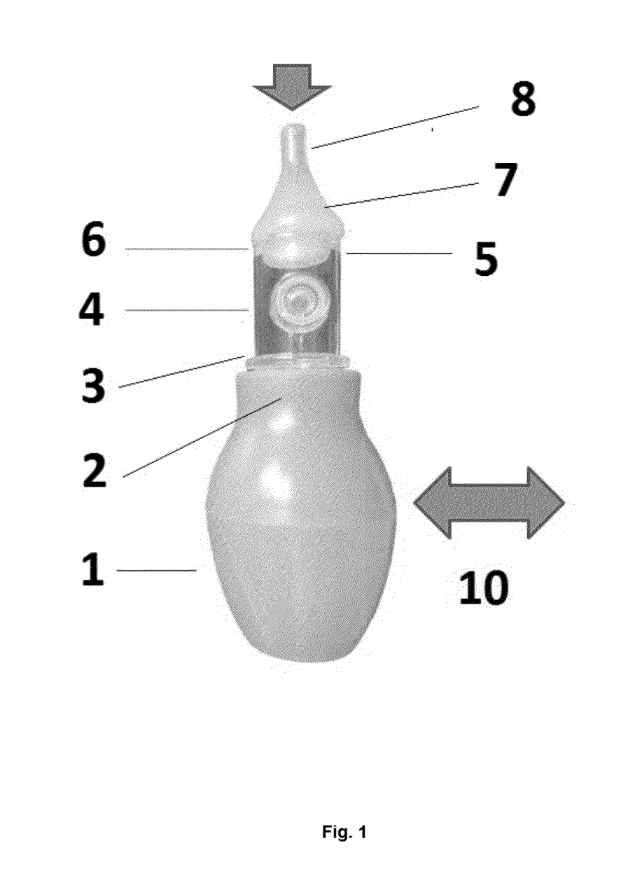

Referring to the drawings, FIG. 1, a device 10 in accordance with the present invention comprises a unique cone-shaped tip 7 and the sharp end 8, a one-way liquid valve 6; a chamber 3; an air valve 4 which is fixed in the chamber to release pressure when the liquid is entered into the chamber; an elastic bulb 1 which is connecting open 2 to the other end of the chamber. When it is squeezed or released, the elastic bulb generates sucking or pushing air pressure to draw the liquid out of the body.

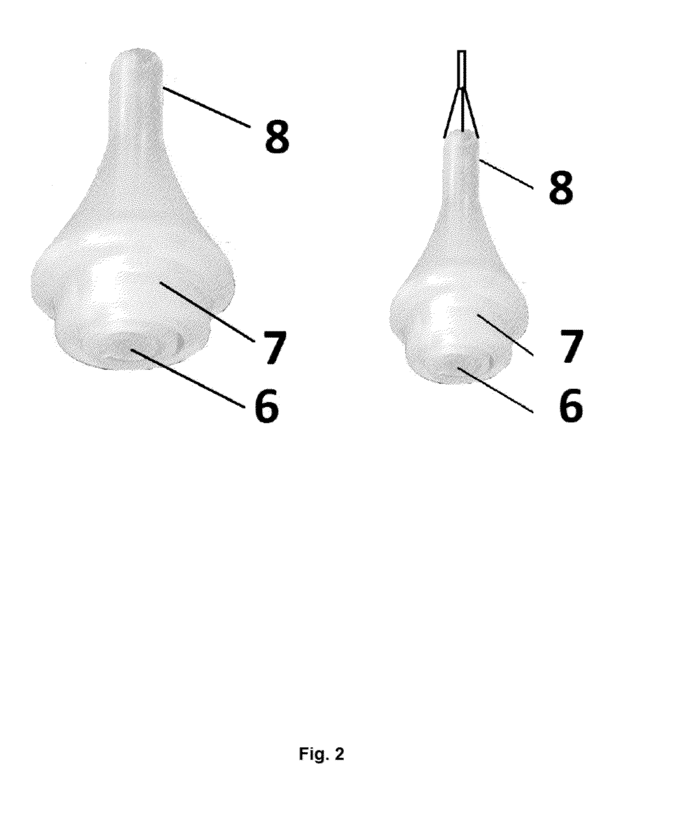

It is further disclosed that the present invention has the following features in FIG. 2: 1) the unique cone-shaped tip can be made as a soft-dull end for suctioning the liquid from an open body space, such as the nasal cavity holding mucus. 2) The unique cone-shaped tip can be made with a hard and sharp end, like a syringe needle, for suctioning the liquid from a closed body space, such as the peritoneal cavity holding ascites. 3) The unique cone-shaped tip can be made with or without a liquid valve. When it is made with a liquid valve, the valve can be placed at the either end of tip, or in any place between the two ends.

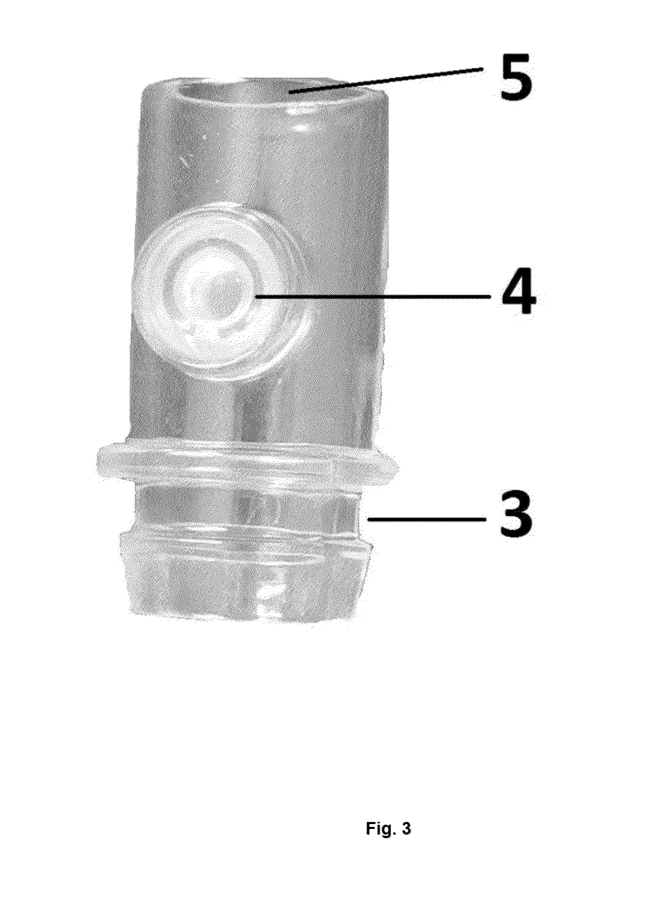

It is further disclosed that the present invention has the following features in FIG. 3: The unique chamber can be made with any safety material, hard or soft It can be equipped with one valve or two valves. When the two valves are equipped, the liquid valve should be placed at the end towards the patient's body space. The air valve can be placed at any part of the chamber's body. Any valve means can be used to form any of the two valves.

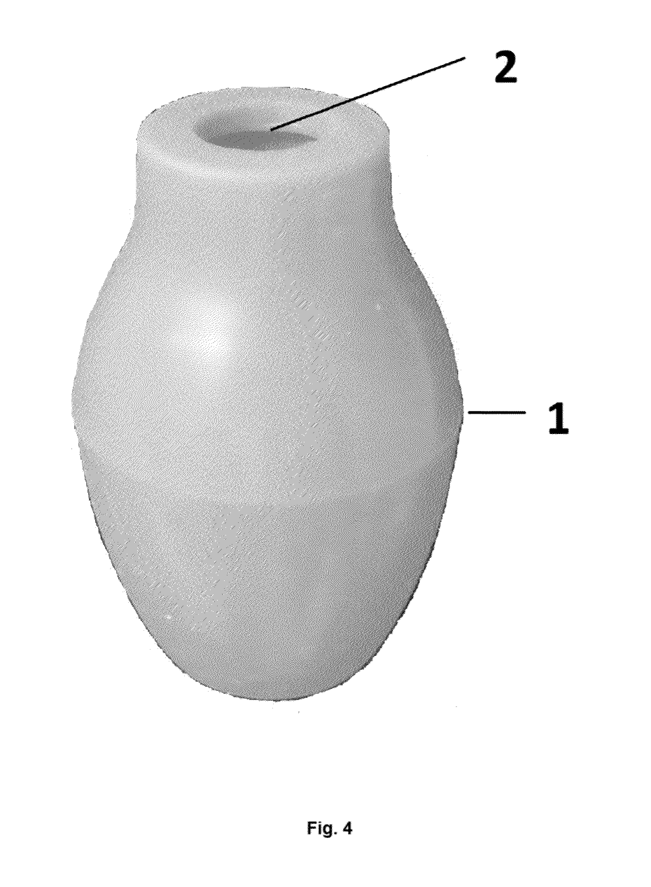

It is further disclosed that the present invention has the following features in FIG. 4: The unique elastic-bulb can be made with any safety materials. Preferably, it is made with silicone material. It can have various holding volumes ranged from 1 ml to 1000 ml. Preferably, it can hold 2-100 ml. More preferably, it can hold 5-50 ml. The elastic-bulb can be made in any regular or irregular shapes.

It is further disclosed that the present invention has the following features: 1) said cone-shaped tip is tightly connecting with the chamber through two matching magnetic surfaces; 2) said cone-shaped tip is tightly connecting with the chamber through a screw connection; 3) said cone-shaped tip is tightly connecting with the chamber through direct contact with one piece inside the other piece; 4) said elastic bulb is tightly connecting with the chamber through two matching magnetic end-surfaces; 5) said elastic bulb is tightly connecting with the chamber through a screw connection; 6) said elastic bulb is tightly connecting with the chamber through direct contact with one piece inside the other piece.

### Description of the Invention of the Method of Use

The present invention further provides a method of treating a disease of a mammal. Using the current invention, a medical professional or a non-medical professional can effectively remove extra mucus from the nasal cavity of a mammal. This device can also be used to remove the effusion in the ear channel of a mammal. The method of use is pretty simple and easy even for a non-medical professional. The user can use one hand to hold the elastic bulb and repeat the action of squeezing-releasing while place the tip inside the space holding the mucus. The mucus will be suctioned into the device. The user is to continue the squeezing-releasing process until no more mucus can be suctioned out. After the excessive mucus is removed, the patient can breathe better.

Using the current invention, a medical professional can effectively remove extra fluid inside the body space, such as ascites in the peritoneal cavity of a mammal. This device can also be used to remove the hydrothorax in the pleural cavity. The method of use is pretty simple and easy for a medical professional. The user can use one hand to hold the device and use the sharp tip to penetrate the skin to let tip be inside of the body fluid. The user can then repeat the action of squeezing-releasing to suction out the fluid. The user is to continue the squeezing-releasing process until no more fluid can be suctioned out. After the excessive body fluid is removed, the patient can have an easy time to breathe (if it is hydrothorax) or the abdomen pain can be alleviated (if it is ascites).

The present invention further provides a method of diagnosing a disease of a mammal. Using the current invention, a medical professional can effectively obtain a special fluid from the nasal cavity or an ear channel of a mammal. The method of use is pretty simple and easy for a medical professional, even for a non-medical professional. The user can use one hand to hold the elastic bulb and repeat the action of squeezing-releasing while place the tip inside the space holding the mucus. The mucus will be suctioned into the device. The user can easily transfer the specimen into a sterile container, such as a sterilized test tube, then send the sample to a clinical laboratory for diagnosis.

Using the current invention, a medical professional can effectively obtain a specimen of the fluid inside the body space, such as ascites in the peritoneal cavity, or the hydrothorax in the pleural cavity, of a mammal. The method of use is pretty simple and easy for a medical professional. The user can use one hand to hold the device and use the sharp tip to penetrate the skin to let the sharp tip be inside of the body fluid. The user can then repeat the action of squeezing-releasing to suction out the fluid. The user can transfer the specimen of either hydrothorax or ascites into a sterilized test tube. The sample is then sent to a clinical laboratory for diagnosis.

### Description of the Invention of the Process

The present invention further provides a process of treating or diagnosing a disease of a mammal. The process is simple and easy to complete. The following steps can be performed by a medical professional or even a non-medical professional.
A. The process of removing excessive liquid from nasal cavity or an ear channel:
   1) Check to make sure all pieces of the anti-backflow device is well assembled;
   2) Check to make sure all pieces of the anti-backflow device is clean before use for removing the body fluid in a non-sterile environment, such as the nasal cavity or an ear channel;
   3) Hold the elastic bulb and place the tip inside the space holding the mucus or effusion;
   4) Repeats the action of squeezing-releasing to suction out the mucus from the nasal cavity or an ear channel;
   5) Observe if no more mucus is left, stop the squeezing-releasing action, and remove the device away from the body space.
   6) Use soap-water to rinse away the mucus from each part of the device after the device is disassembled; If the device is disposable, then just throw the whole set into a container to hold biohazard materials;
   7) Air dry the device for the next use.
B. The process of removing the body liquid from inside the body space, such as such as ascites in the peritoneal cavity, or the hydrothorax in the pleural cavity:
   1) Check to make sure all pieces of the anti-backflow device is well assembled;
   2) Check to make sure all pieces of the device is clean and sterilized before use for diagnosis of a disease and for removing the body fluid within the body space, such as ascites in the peritoneal cavity, or the hydrothorax in the pleural cavity;
   3) Hold the elastic bulb and penetrate the skin by using the sharp tip to place the tip inside the space holding ascites or hydrothorax;
   4) Repeats the action of squeezing-releasing to suction out ascites or hydrothorax from the body space;
   5) Observe if no more ascites or hydrothorax is left, stop the squeezing-releasing action, remove the device away from the body space;
   6) Use soap-water to rinse away ascites or hydrothorax from each part of the device after the device is disassembled; thoroughly clean the device before sterilization. If the device is disposable, then just throw the whole set into a container to hold biohazard materials;
   7) Sterilize the cleaned device for the next use.

The following examples further illustrate the present invention but, of course, should not be construed as in any way limiting its scope.

### EXAMPLE 1

This example illustrates the efficacy of the mucus extractor in removing excessive mucus from the nasal cavity. The mother was holding her 2-year old son with her left arm and holding a mucus extractor with her right hand. She placed the soft tip of the mucus extractor slightly inside of her baby's right nostril. Then she repeated the action of squeezing and releasing the elastic bulb. She saw some mucus was drawn into the mucus extractor. She continued the process for a few minutes. After she saw there was no more mucus coming out, she then stopped the process. She held the baby for a while and felt with her hand that her baby had a nasal air movement. After laying her baby in the bed, she took the mucus extractor to a bathroom to clean the mucus extractor with soap and water.

### EXAMPLE 2

This example illustrates the procedure of obtaining a biological specimen from a covered body space. A laboratory scientist used the extractor with a hard-sharp tip to perform an experiment. He filled a cleaned rubber ball with special saline solution containing potassium chloride at 1% concentration. The scientist used his left hand to hold the rubber ball, and used his right hand to hold the Extractor. He inserted the sharp tip of the Extractor into the rubber ball, then squeezed and released the elastic bulb of the Extractor. After getting about 2 ml of the solution, he transferred the solution into a clean test tube. Then he performed a lab test to determine the concentration of potassium in the specimen. The result matched almost exactly what the concentration of the solution he made. This simple experiment indicated that the Extractor can be used to obtain the biological sample from an closed space, like a mammal's body.

### EXAMPLE 3

This example illustrates the usefulness of using the present invention to remove abnormal body fluid for relieving the pain. A medical doctor designed a test model: When the rubber ball was fulfilled with enough liquid, the alarm will be triggered. When the liquid was drawn, the inner pressure within the ball is reduced, the alarm will stop. He used his test model to determine if the Extractor can be used to remove the body fluid. He inserted the sharp tip of the Extractor into the rubber ball, then squeezed and released the elastic bulb of the Extractor. More and more liquid was drawn from the ball. The surface pressure of the ball was reduced gradually. After the liquid was removed for two minutes, the alarm was stopped. This simple experiment indicated that the Extractor can be used to remove the fluid from a closed body space as therapy.

Through the easy use of the Extractor 4 in the above mentioned situations, the general population now has a simple, safe, easy and inexpensive method that can be used to remove a number of abnormal liquids from the body to treat and prevent a variety of the human diseases. This system can also be used to obtain specimen for diagnosis of a disease. The significant advantage of using this new system is that there will be less chance to contaminate the human body space, reduce the pain quickly and help to regain the capacity of nasal breath. In addition, the medical community now has a simple and reliable method and process to diagnose and treat certain diseases.

Those skilled in the art will appreciate that changes and modifications of the Extractor can be made, as well as the specific process and the methods of use disclosed herein without departing from the spirit and scope of the present invention as set forth as defined by the following claims.

What claimed is:

1. An extracting device for diagnosis and treatment of a disease comprising:
(1) a cone-shaped tip with a central inner hole through which the liquid material can flow through;
(2) a one-way liquid valve fixed at the bottom of the e cone-shaped tip or at the one end of the chamber;
(3) a chamber which directly connects to the bottom of the cone-shaped tip;
(4) a one-way air valve which is fixed on the chamber, and
(5) an elastic bulb which directly connects to the other end of the chamber for generating suctioning or pushing pressure when it is squeezed or released.

2. The extracting device for diagnosis and treatment of a disease as claimed in claim wherein said cone-shaped tip is smooth and is made with soft material.

3. The extracting device for diagnosis and treatment of a disease as claimed in claim 1, wherein said cone-shaped tip is sharp and is made with hard material.

4. The extracting device for diagnosis and treatment of a disease as claimed in claim 1, wherein said chamber holds at least 0.5 ml of liquid.

5. The extracting device for diagnosis and treatment of a disease as claimed in claim 1, wherein said elastic bulb holds at least 0.5 ml of liquid.

6. The extracting device for diagnosis and treatment of a disease as claimed in claim 1, wherein said cone-shaped tip and said chamber can be connected tightly with the magnetic force.

7. The extracting device for diagnosis and treatment of a disease as claimed in claim 1, wherein said cone-shaped tip and said chamber can be connected tightly with the mean of screw.

8. A process of using the extracting device for diagnosis and treatment of a disease comprises:
(1) a user to select an extracting device with a cone-shaped tip with a central inner hole through which the liquid material can flow through; a one-way liquid valve fixed at the bottom of the cone-shaped tip; a chamber which directly connects to the bottom of the cone-shaped tip; a one-way air valve which is fixed on the chamber, an elastic bulb which directly connects to the other end of the chamber for generating suctioning or pushing pressure when it is squeezed or released;
(2) a user to insert the tip into an open body space to remove the excessive body fluid, such as nasal secretion, to regain nasal breathing;
(3) a user to insert the tip through penetration into a close body space to obtain body fluid as a specimen for a laboratory test to diagnose a disease, such as a nasal secretion specimen for testing influenza, or as a way of removing excessive body fluid for a treatment, such a removing ascites, or similar.

9. The process of using the extracting device for diagnosis and treatment of a disease as claimed in claim 8, wherein said user is a medical professional.

10. The process of using the extracting device for diagnosis and treatment of a disease as claimed in claim 8, wherein said user is a non-medical professional.

11. A method of using the extracting device for diagnosis and treatment of a disease comprises:
(1) a user to choose an extracting device which comprises a sharp cone-shaped tip with a central inner hole through which the liquid material can flow through; a one-way liquid valve fixed at the bottom of the cone-shaped tip; a chamber which directly connects to the bottom of the cone-shaped tip; a one-way air valve which is fixed on the chamber, and an elastic bulb which directly connects to the other end of the chamber for generating sucking or pushing pressure when it is squeezed or released to treat a disease by removing unwanted fluid out of the body.

12. The method of using the extracting device for diagnosis and treatment of a disease as claimed in claim 11 wherein said user is a medical professional.

13. The method of using the extracting device for diagnosis and treatment of a disease as claimed in claim 11, wherein said user is a non-medical professional.

14. The method of using the extracting device for diagnosis and treatment of a disease as claimed in claim 11, wherein said unwanted fluid is nasal secretion.

15. The method of using the extracting device for diagnosis and treatment of a disease as claimed in claim 11 wherein said unwanted fluid is sputum in the patient's mouth.

16. The method of using the extracting device for diagnosis and treatment of a disease as claimed in claim 11, wherein said unwanted fluid is ascites.

17. The method of using the extracting device for diagnosis and treatment of a disease as claimed in claim 11, wherein said unwanted fluid is hydrothorax in the pleural cavity.

18. The method of using the extracting device for diagnosis and treatment of a disease as claimed in claim 11, wherein said unwanted fluid is pleural effusion accumulated between the two pleural layers.

19. The method of using the extracting device for diagnosis and treatment of a disease as claimed in claim 11, wherein said unwanted fluid is nephrohydrops.

20. The method of using the extracting device for diagnosis and treatment of a disease as claimed in claim 11, wherein said unwanted fluid is ear effusion.
